(19) United States
(12) Patent Application Publication
(10) Pub. No.: US 2009/0069214 A1
(43) Pub. Date: Mar. 12, 2009

(54) Modulator of Metabolism Processes and Method of Its Receiving

(76) Inventor: Gennadiy Gavrilovich SHITOV, Kharkov (UA)

Correspondence Address: BEEM PATENT LAW FIRM, 53 W. JACKSON BLVD., SUITE 1352, CHICAGO, IL, 60604-3787, US

(21) Appl. No.: 12/208,607

(22) Filed: Sep. 11, 2008

(30) Foreign Application Priority Data
Sep. 11, 2007 (RU) RU2007133960

(51) Int. Cl. A61K 38/00

(52) U.S. Cl. 514/2

(57) ABSTRACT

A modulator of metabolic processes and method of manufacturing same is provided. The invention may have applicability in at least the fields of medicine, veterinary medicine, medical industry, and agriculture. The essence of invention is a chemically modified biopolymer with a polypeptide chain as an exogenous modulator of metabolic and energy exchange processes between a cell and environment, characterized in that it comprises chlorine atoms covalent-bonded with nitrogen atoms of polypeptide chain to the amount of 0.5 to 40% of the total mass of modified biopolymer, whereas the polypeptide chain includes methionine sulfoxide, cysteinic, α-aminomalonic and α-aminoacetoacetic acids to the amount of 1-20 acid residues per 1 mole of final product. The proposed modulator possesses large resource of free energy due to high degree of oxidation, thus ensuring catalytic activity when interacting with clue enzymes and return of particular organs and systems to normal physiological condition.

### CROSS-REFERENCE TO RELATED APPLICATIONS

This application claims the benefit of priority from Russian patent application number RU 2007133960, filed Sep. 11, 2007.

### BACKGROUND OF THE INVENTION

The present invention relates to the field of biology and may be applied in medicine, veterinary medicine and agriculture for correction and regulation of metabolic processes in order to maximize vitality of human, plant and animal organisms and to maintain their normal physiological condition.

Unfavorable external and internal factors well known from literature alter the properties of biomolecules, which leads to violation of natural metabolism and energy exchange between a cell and environment and to appearance of pathological conditions in various organs and systems (diseases). Therefore, it is a universally recognized truth that all pathologies in any organism are consequences of violated metabolism, and this present a highly difficult problem in medicine and biology.

The object of the present invention is to eliminate this problem and to find one of possible options of its practical solution.

### DETAILED DESCRIPTION OF THE INVENTION

In spite of complicated character of regulatory system, all central metabolic ways have been completely revealed by this day; still, the mechanisms of their regulation remain unknown. This, in turn, is caused by lack of sufficient information on metabolism regulation in cells of living organisms at molecular level; therefore, it would take much time to perform necessary research (P.Mapp [symbol] p., [symbol] ox [symbol] [symbol] oBeKa, M., M [symbol] p, 2004).

By generalization, systematization and scientific interpretation of the results of research in mechanism of natural regulation of enzymatic reactions proceeding in bacterial cells the basic principle underlying this process was found to be a change in catalytic activity of one or several clue enzymes of metabolic fate. Biochemical compounds that increase catalytic activity were named positive modulators (P. Mapp [symbol] p., [symbol] ox [symbol] [symbol] oBeKa, M., M [symbol] p, 2004) or effectors (Biochemistry, edited by E. S. Severin, Moscow, GEOTAR Media Publishers, 2000, in Russian).

It should be noted that the biochemical processes connected with alterations in catalytic activity of enzymes are endogenous, thus, they pass outside the human will. Biochemical products participating in such processes are metabolites of cellular exchange, and their composition and structure are not clear yet.

Scientific literature and patents contain no information relating to exogenous bioregulators. We suppose this is due not only to extreme complexity of mammalian metabolism and its regulation, but also to insufficiency of proven concepts of regulatory process, particularly, their mechanisms, taking place in cells of living organism tissues. It is extraordinarily difficult to substantiate, even as a hypothesis, chemical composition and structure of a modulator in such a way that exogenous action on the course of metabolic processes became possible.

From the other side, it is also evident, that any protein product on the basis of natural fragments—amino acids, if introduced to an organism would cause an inevitable response: it would be met as “alien”, i.e., its proteolysis and usage as a building material with possible cytotoxic reactions would follow.

Immune system protects individuals from alien antigens and, when meeting them, responds by activation of specific cells, such as T- and B-lymphocytes, and by production of soluble factors like interleukins, antibodies and complement factors. Antigen to which immune system responds is degraded by antigen-presenting cells (APCs), and the antigen fragment connected with Class II glycoprotein of main hystocompatibility complex (MHC) is expressed on cell surface. MHC complex glycoprotein-antigen fragment is presented to T-cells who by their T-cell receptors “recognize” antigen fragment together with Class II MHC protein with which they are connected. T-cell becomes activated, i.e., it proliferates and/or produces interleukins, which brings to expansion of activated lymphocytes aimed at the antigen—object of immune attack (Grey et al., Sci.Am., 261: 38-46, 1989). Thus, it may be theoretically possible to create modulators of metabolic processes on the basis of natural protein fragments, but in practice it would be highly dubious due to high immunological protection of organism. Accordingly, any research aimed at establishment of long-action regulatory peptides has not brought any results. This is due to the fact that immunological and allergological properties of final products may are unpredictable because of various types of antigen specificity (species, stage, organ and organoid specificity). A substantial obstacle in creation of regulatory peptides is their quick decay, irrespective of type of administration, and impossibility of oral administration (.M.Ashmarin, Prospects of Practical Application of Regular Peptides and Certain Fundamental Studies, Vopr.Med.Khim., 1984, Vol. XXX, #3). This circumstance emphasizes the necessity of special method of their establishment.

A promising way in purposeful search of such substances may be synthesis of polypeptides—inductors of immune system tolerance to various antigens causing certain diseases (HIV, malignant tumors, etc.) by revelation of peptide structure in respective receptor and subsequent synthesis with an identical amino acid sequence (RU 2215005 C2 of 27.10.2003; RU 2223290 C2 of 27.04.2004; RU 2199548 C2).

In spite of positive results of clinical tests of the patented peptides on several volunteers, the problem of their metabolism remained unclear, as despite their “affinity”, they remain unidentified antigens for the organism and thus, to our opinion, must inactivate quite soon. The speed of inactivation as well as of adaptation of organism to such inductors is determined not only by organism vitality level, but also by various factors, such as inevitable hydrolysis due to endo- and exopeptidases. Nevertheless, the main limiting factor of their wide application in medical practice is unexplored metabolism of such modulators, thus, the distant consequences remain unpredictable, as the nature may not be cheated. That is why the described methods of metabolism regulation are too particular and cannot be treated as a general method for violated natural metabolism correction, with due consideration of more than 10,000 nosological units (types) of pathologies existing with many clinical variants each.

Vain results of application of such peptides in such conditions, as “irreversible pathology”, “dystrophy”, “atrophy” and the like, are manifest.

The present invention solves the discussed problem by practical realization of theoretically substantiated author's concept of development of drugs for regulation and correction of metabolic processes as published in Vestnik Ross.Akad.Med. Nauk, 1992, #5, pp. 26-30.

The basic clauses of this concept with some corrections are disclosed further.

As discovered by Russian physiologist P. K. Anokhin (Anokhin P. K., Critical Questions of Functional System Theory, Moscow, 1980, in Russian), any organism should be treated as a functional system based on self-regulation. The principle of self-regulation means that any deviation of the result of functional system activity from the level determining normal (natural) metabolism or other aspects of normal vital activity on the basis of feedback does immediately selectively mobilize various mechanisms of the system in order to return this result to optimal metabolic level. Still, under certain environmental effects the system does not return to optimal level, hence, the violation of metabolism results in various pathological conditions.

However complicated the regulatory system is, all central metabolic ways have been almost all found, still, their regulation mechanisms remain unexplored (H [symbol] cxo [symbol] M [symbol] , CTapTK, Pery [symbol] MeTa [symbol] 3Ma, M., M [symbol] p, 1977). Thus, the clue to solution of problem of really efficient drugs of any pharmacotherapeutic group lies in revelation of metabolism regulation mechanism. As this problem is too difficult in practice, an alternative option or, more exactly, another engineering approach to its solution based on the following well-known principles of biochemistry, biotechnology and pharmacy. First, the drug must be proteinic in nature, as all biochemical processes, including metabolism and its regulation, take place with proteins. Second, the initial raw material must be as species compatible as possible, i.e. the closer the proteinic substance is to human nature, the more attainable becomes the purpose (the best option is usage of human organs and tissues, such as placenta). Third, the velocity of biochemical reactions depends not on the law of acting masses, but on the activity of key enzymes. Fourth, natural biochemical products of animal origin obtained from organs and tissues by extraction and separation cannot ensure effective treatment and reproducibility of results in both manufacturing standard and pharmacological activity due to species incompatibility (antigens) and permanently variable composition and structure. Fifth, the necessary effect of metabolism drug regulation (correction) may be achieved only in case when sufficient stability of reactant(s) is ensured both within and outside the system of organism. This is one of the most important biochemical and engineering requirements, because any natural regulator, even when its structure is determined and synthesis performed would inevitably inactivate after administration.

One of substantial ways of alteration of enzyme activity or, respectively, of biochemical reaction velocity is their interaction with certain signaling biochemical products. This enables recognition of various signals and integration of obtained information, thus switching a metabolic path on or off. In case of a pathological condition “forced” introduction of a substance or a sum of substances into a dynamic self-regulating system able of selectively connect various organs and levels of nervous and humoral integration, as a regulator able of only activation, not deactivation, of a metabolic way with simultaneous acceleration of slowed biochemical reactions, permits restoration of partially or totally lost physiological functions of system tissues.

Such regulatory products may be proteinic substances—fragments of macromolecules, containing some oxidized functional groups that are generally absent under normal natural physiological condition. This is the essence of the concept of purposeful search for novel generation drugs that was formulated before on the basis of systematization, generalization of literary data and the results of own research (Shitov G. G. et al., Khim-Farm.prom. Obzomaya informatsia.—M., 1989—Issue 3). The premises forming the basis of this concept will be discussed further.

First, thermodynamic force of any biochemical process is determined by value of energy released in hydrolysis of special donor substances in a high degree of oxidation. Thus, such an energy-carrying substance is adenosine triphosphate (ATP) containing two phosphorus-anhydrile connections corresponding to maximum phosphorus oxidation. I.Kloiz ( [symbol] ., [symbol] HepreT [symbol] Ka [symbol] ox [symbol] ecK [symbol] x peaK [symbol] ,M., 1979) states that actual value of energy released in hydrolysis under normal cell conditions is about 12 ccal/mole. Energy release in ATP hydrolysis ensures the course of thermodynamically unfavorable processes and shifts coupled reaction equilibrium approximately 108 times. Similarly, the thermodynamic moving force in the process of electron reception-transfer during oxidation of fuel molecules is the energy depending on oxidation degree of nicotinamide adeninedinucleotide phosphate (NADP) and nicotinamide adeninedinucleotide (NAD).

When this pattern of metabolism is used for development of drugs, it becomes quite evident that the drug must also possess a sufficiently expressed thermodynamic activity, i.e. be in oxidized state and serve as a certain source of energy for energetically inefficient reaction with enzymes. Degree of oxidation of a protein or polypeptide molecule may be increased by introduction of additional oxygen atoms (hydroxyl or carbonyl groups, sulfons, etc.)

Second, oxidation.(hydroxylation) of amino acids in organism must be a special protective step as evident from multiple publications telling of increasing hydroxyproline and hydroxylysine content in blood serum or urine under many pathological conditions (Komarov F. I. et al., Klin.Med., 1977, #11, pp. 62-67). In this case we speak about interpretation of those data. Is increased hydroxyproline content a violation of metabolism (pathological decay of tissues) or, visa versa, a purposeful protective action? The latter option is supported not only by the facts of its increase in almost all kinds of pathologies or under normal course of pregnancy, but by some special research as well. If a 14C-tagged hydroxyproline or 14C-hydroxylysine is administered to a rat, no radioactivity is observed in newly synthesized connective tissue protein (collagen), whereas after administration of 14C-proline the collagen shows radioactivity (Cardinale G., Enzymol., 1974, vol. 41, pp. 245-300). At the same time it is not yet clear why the content of oxidized amino acids in blood or urine increases under pathologies, if proline cannot be hydroxylyzed outside the biopolymer. Consequently, connective tissue proteins or other protective proteins are additionally oxidized in pathological conditions, their fragments are thrown into the system, and those fragments may be treated as endoregulators under a pathological process. The above facts are indicative of a special role of highly oxidized amino acids in increased resistance of organism to harmful factors and support a supposition that inclusion of hydroxylized acids into a protein or polypeptide may bring both to reduction of immunodeficiency and to acceleration of biochemical reactions aimed at metabolism correction.

Third, one of substantial premises for formation of the concept discussed is result of study in chemical structure of fibrillary proteins (collagen) contained in skin, bones, tendons, cartilage, teeth, etc. Collagen has a unique structure and unique sequence of amino acids. Proline content is much higher in collagen than in other proteins, thus facilitating hydroxylation. Besides, collagen contains oxidized amino acids, for instance, in basal membranes 6%, in hyaline cartilage 2.0-2.5% (CTpa [symbol] ep [symbol] ., [symbol] ox [symbol] , B 3 TT., M., 1985). Under irradiation and thermal action the content of hydroxylized proline and lysine in collagen substantially increases, therefore, the process of amino acid oxidation correction mediated by prolinehydroxylase (CTpa [symbol] ep [symbol] ., [symbol] ox [symbol] , B 3 TT., M., 1985) in both collagen and other protective proteins is critical for increase of organism resistivity.

Fourth, a special theoretical interest lies in metabolism of oxidized biopolymers. Metabolism of particular drugs—xenobiotics—has been studied to a sufficient extent and in some cases harmful effect of their metabolites on protective systems has been proved, especially after prolonged administration. In other cases metabolites are main active factors. A natural biopolymer, if oxidized to a higher degree, is transferred to metabolic state. Still, unlike an antibiotic, it would not be removed from organism in such state, as this state is a form of existence for proteinic bodies. A further metabolism of an oxidized biopolymer is likely to proceed similarly to endoregulators, i.e. via hydrolysis into small fragments, as will be shown further. The products of such enzymatic hydrolysis become main active factors.

Thus, the proposed concept of purposeful search for drugs may also be treated as a concept of drug metabolic regulation under immunodeficiency and similar pathological conditions.

For practical realization of this concept a substantiation of process techniques and methods was necessary enabling to perform the process of biopolymer chemical modification, meeting all terms as specified above.

The choice of human placentary tissues as the main source of biopolymers is based on the fact that they were ideal objects for attainment of our purposes. By this time a large number of works has been published devoted to usage of placenta for manufacture of miscellaneous drugs. A broad pharmacological spectrum and high therapeutic effect of their administration bring actually unlimited prospects in development of drugs, including those able to regulate (correct) violated metabolism.

Issuing from the presented substantiation, a condition sine qua non for development of metabolic process modulators is usage of species compatible biopolymers by way of their special chemical treatment. For instance, corresponding biopolymers contained in placentary tissues for mammalia, biopolymers of floral origin for plants, etc.

In accordance with the formulated object the final product after chemical modification of biopolymers must have such physico-chemical and biochemical properties: (1) availability of peptide bonds; (2) high bioavailability and biocompatibility; (3) absence of antigen properties; (4) the highest possible degree of oxidation.

Biopolymers of the same species as initial raw material predetermine high bioavailability, biocompatibility and enable preservation of peptide bonds. Still, all biopolymers in native form irrespective of their homogenization degree, thermal incubation, fractionization, etc. express immunogen properties, therefore, in order to obtain final products with absent antigen properties they must exist in oxidized, i.e. metabolic, state, typical for natural endoregulators that contain one or more amino acids in oxidized form, like oxyproline, oxylysine, etc. Under natural conditions endoregulators having performed their function are proteolytically splitted, and amino oxyacids formed in this way are not used as “building materials”, but removed with urine.

Biopolymer oxidation substantially alters physico-chemical and biochemical properties of the final product due to increased free energy, which in turn leads to increased modulator reactivity during interaction with enzymes. Thus, coupling of an oxidized biopolymer fragment (modulator) to an enzyme with large resource of free energy would mean increase of catalytic activity of the clue enzyme in metabolic process. From the other side, oxidized state of modulator substantially reduces the velocity of its further metabolism and respectively prolongs the period of its biological effect.

For practical realization of this idea applicant with his co-workers studied the process of chemical modification of various biopolymers using chlorous acid as a reagent possessing high selectivity in hydrolysis of peptide-glycoside bonds and in oxidation of particular functional groups in biomolecules. High selectivity of chlorous acid is caused by its instability:

9 HClO2→3 HCl+H2O+3 ClO2+1.5 Cl2

Formation of a covalent nitrogen-chlorine bond causes an integration of nitrogen-carbon bond, thus determining its high resistance to proteolysis which increases due to impossibility of an enzyme-substrate complex.

Our research could find out that in a polypeptide chain only serine, trionine, methionine, cystine and cysteine are oxidized. All amide- and amine-containing amino acids suffer chemical transformation due to emerging covalent nitrogen-chlorine bonds. Thus, in the final product obtained by a biopolymer chemical modification, glycine, alanine and phenylalanine remain unaltered, and they were identified by amino acid analyzer. We found that seribe within the polypeptide chain is oxidized to α-aminomalonic acid, whereas triptophane and tyrazine are completely destroyed, that is, the polypeptide chain is broken at the place of their location, which is confirmed by data from literature (Proteins: in 4 vols, edited by P. Neuroth and C. Belly, Vol. 1, Chemistry of Proteinic Substances, pp. 130-135, 173, 186,281-290—in Russian).

Sulfur-containing amino acids (cystine and cysteine) are easily oxidized by hypogalogenites to cysteinic acid, while methionine oxidizes to a corresponding sulfide (M. R. F. Asworth, Tytrimetric Methods of Organic Analysis. Direct Titration Methods, Moscow, 1968, 1968, pp. 219-223—in Russian). We proved the presence of cysteinic acid-characterizing sulfogroups in the polypeptide by IR-spectral valency fluctuations near 1670 cm-1 and performed their quantitative definition as per (Kullbom S. D., Smith H. F.//Analytical Chemistry—1963, 35, p. 1005).

The process of chemical modification of polypeptide chain-containing biopolymers may be expressed by the following reaction equations:

a) chloination of peptide bond

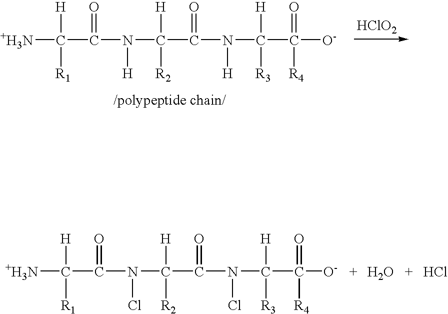

δ) oxidation of amino acids in the biopolymer.

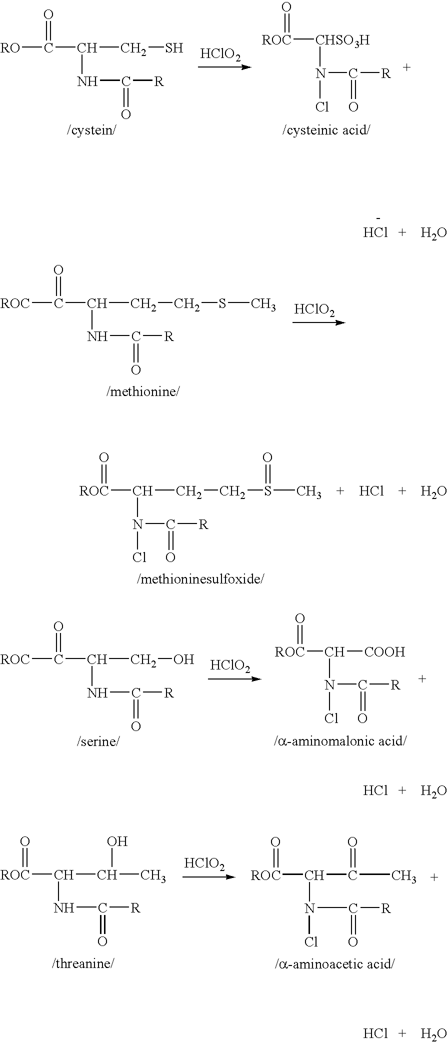

Amino acid composition of the obtained modified biopolymer is specified in Table 1.

TABLE 1
TABLE 1: Amino acid composition of the modulator

| | | | | Amino acid | | | |
|---|---|---|---|---|---|---|---|
| | | | | residue content | | | |
| | | | | Molecular | | Chlorine content | |
| # | Amino acid | units | Formula | mass | % | mass | % |
| 1 | Chlorinated amino acids | | | | | | |
| 1.1 | Glycine | 17 | C₂H₂ClNO | 1190.41 | 8.6 | 380.8 | 31.98 |
| 1.2 | Alanine | 2 | C₃H₄ClNO | 211.18 | 1.53 | 73.06 | 34.50 |
| 1.3 | Valine | 2 | C₅H₈ClNO | 267.3 | 1.9 | 71.075 | 26.59 |
| 1.4 | Leucine | 1 | C₆H₁₀ClNO | 147.67 | 1.06 | 36.33 | 24.6 |
| 1.5 | Isoleucine | 2 | C₆H₁₀ClNO | 147.67 | 1.06 | 35.5 | 24.04 |
| 1.6 | Histidine | 2 | C₆H₄Cl₂N₃O | 343.32 | 2.48 | 86.13 | 25.09 |
| 1.7 | Proline | 6 | C₅H₆ClNO | 1053.04 | 7.63 | 320.4 | 30.43 |
| 1.8 | Lysine | 2 | C₆H₉Cl₃NO | 594.57 | 4.31 | 291.52 | 50.74 |
| 1.9 | Phenylalanine | 3 | C₉H₈ClNO | 545.07 | 3.95 | 106.61 | 19.56 |
| 1.10 | Asparagine | 5 | C₄H₃Cl₃NO | 728.48 | 5.27 | 313.78 | 43.07 |
| 1.11 | Aspartic acid | 2 | C₄H₃ClNO | 448.8 | 3.25 | 136.76 | 30.47 |
| 1.12 | Glutamine | 8 | C₅H₅Cl₃NO | 1569.2 | 11.37 | 735.38 | 46.86 |
| 1.13 | Glutamic acid | 6 | C₅H₆ClNO | 654.52 | 4.74 | 176.7 | 27.01 |
| 1.14 | Arginine | 8 | C₆H₇Cl₅NO | 3558.92 | 25.78 | 1904.92 | 53.5 |
| 2. | Oxidized amino acids | | | | | | |
| 2.1 | Methionine → | 2 | C₄H₈Cl₃NOS | 288.42 | 2.09 | 136.82 | 47.43 |
| | methioninesulfoxide | | | | | | |
| 2.2 | Threonine → | 3 | C₄H₄Cl₃NO₂ | 403.73 | 2.95 | 210.25 | 52.07 |
| | α-aminoacetic acid | | | | | | |
| 2.3 | Serine → | 3 | C₃H₅Cl₃NO₂ | 406.77 | 2.96 | 208.35 | 51.22 |
| | α-aminomalonic acid | | | | | | |
| 2.4 | Cystine → | 4 | C₄H₄ClNO₄S | 918.25 | 6.65 | 165.09 | 17.97 |
| | Cystic acid | | | | | | |
| 2.5 | Cysteine → | 4 | C₃H₄ClNO₄S | 918.25 | 6.65 | 165.09 | 17.97 |
| | Cysteinic acid | | | | | | |
| | Total | 82 | | 13801.00 | | 5169.52 | 37.45 |

Maximum possible chlorine content per 1 mole of the modulator is 37.45%, whereas the upper limit of content in this application is claimed to be 40%, the lower limit being 0.5%. The content of oxidized amino acids per 1 mole of the modulator is 2 to 5 residues, the claims herein envisage the range 1 to 20.

Using ion exchange chromatography, from products of reactions between biopolymers and chlorous acid we extracted two polypeptide fractions with average molecular mass 13,800 and 14,200 D by Lowry. Polypeptide chain consists of 82 and 86 amino acid residues respectively. The first fraction, as IR spectroscopy shows (Kullbom S. D., Smith H. F., Analytical Chemistry, 1963, 35, p. 1005) contains sulfogroups to the amount 0.6% of polypeptide mass as per sulfur, whereas in IR spectrum of the second fraction valence fluctuations in 1670 cm-⁻¹ area are absent. Polypeptides contain chlorine 35.6% and 36.5% respectively.

Thus, our final product is a chlorinated highly oxidized polypeptide, wherein combustible part (carbon plus hydrogen) makes only 32% of total mass. Addition of chlorine atoms to the polymer means its transfer to a still higher degree of oxidation. Each chlorine atom lends one electron to the polymer, increasing its free energy to as high level as possible. Such polymer chemical modification defines unique physico-chemical, biochemical and pharmacological properties that were not known before and that are characterized by such parameters:
1) Polypeptide is exceptionally resistant to acid and alkaline hydrolysis. After heating of the drug in a sealed ampoule at 100° C. in a solution of hydrochloric acid only glycine, phenylalanine and alanine may be identified.
2) The drug is highly resistant to enzyme hydrolysis if administered via gastrointestinal tract, as its pharmacological activity is actually identical to that of injection form.
   High proteolytic resistance is caused by a covalent chlorine-nitrogen bond, which leads to coupling of a nitrogen-carbon bond.
3) Freezing point of 1% aqueous solution of the drug is −70° C., which is connected to high surfactance.
4) A sterile aqueous solution in ampoules (no fungal spores) preserved its physico-chemical and pharmacological properties for more than 10 years (if stored without direct sunlight). This is due to high degree of oxidation. Oxygen contained in aqueous solution cannot oxidize the drug as well as water cannot hydrolyze it irrespective of the duration of action.
5) It is not toxic. A conditional therapeutical dosage for humans is 0.7 mg/kg. The drug was administered to animals in dosage 100 times above the therapeutical value for 6 months, and none of them died.
6) It shows a completely novel adaptogenic effect on thermal, mechanical overloads and to action of poisonous and toxic substances, including bites of insects, snakes, etc.
7) The drug shows multifunctional pharmacological effect (immunotropic, reparative, anti-inflammatory, resorptive, lactogenous, anti-oxidant, chondroprotective, anti-stress, anti-mutagenic, anti-shock, anti-coagulant, fertile effect, including the treatment of erectility dysfunction and prophylaxis of miscarriages).
8) It possesses quite novel high biological activity as a prophylactic drug for most known diseases, including increased life expectancy and prevention of premature senility (life duration of mice with the drug being administered to the amount of 1 mg/kg for 30 days increased 1.5 times at average).
9) Prolonged pharmacological effect after finished course of treatment (the pharmaco-therapeutical result preserved for 5-6 years).
10) Organism does not adapt to the drug after prolonged administration.
11) The drug is highly effective as a plant growth and development regulator (yield of most cereals increases 1.5-2 times, the plants and their fruits becoming more resistant to harmful environmental factors).
12) The drug is highly efficient in apiculture, mushroom planting, sericulture, animal breeding, mariculture, etc.

Those pharmacological properties and degree of efficiency in each category have been established on the basis of results of pre-clinical and clinical tests performed at some leading research institutes and hospital establishments in Ukraine and Russia.

High pharmacological efficiency of the proposed preparation (hereinafter Drug) was established in laboratory tests on animals (mice, rats, rabbits, dogs), in clinical tests on human volunteers and as a result of post-clinical administration. The following text specifies some results of pre-clinical, clinical and post-clinical administration of the proposed drug in medicine, veterinary medicine and agriculture.

Immunotropic effect: Immunitropic activity of proposed drug was evaluated by standard methods as recommended by World Health Organization (WHO). Specific pharmacological (immunological) activity was tested in model systems that give q quantitative characteristic of the effect of drug tested on the basic immunity links. The tests were performed on guinea pigs, Wistar rats, outbreed mice and mice of CBA line, 357B1 (CBAXC57B1)Γl. The effect of proposed drug was tested on cellular and humoral immune reaction (phagocytic activity, effect on the result of generalized pseudomonas infection, on complement system, on cooperation between T and B lymphocytes, on transplant versus host response, on delayed-type hypersensitivity response, on humoral response). Reference drugs were such known pharmaceuticals as levamisol, T-activine, cyclophosphamide, sodium nucleate, amniocene, pyrogenal.

Test results showed that the proposed drug was substantially more active than the specified reference preparations, only in some experiments being identical to T-activine. The most typical parameter characterizing immunotropic (immunomodulating) activity of any pharmaceutical is evaluation of its effect on delayed-type hypersensitivity (DTH) response. In studies of DHT induction to sublethally irradiated mice (ionizing radiation increases immunological competence of organism by suppressing DTH expression) the proposed drug was administered subcutaneously 5-fold to total dosage 0.4 and 0.6 mg/kg, beginning from the next day after irradiation. As seen from Table 2 below, treatment of animals to dosage of 0.4 mg/kg increases hypersensitivity response by 30% as compared to irradiated mice of control group. At dosage of 0.6 mg/kg response index becomes actually equal to that of non-irradiated animals, which is a demonstration of possibility of organism protection under radiation effect.

TABLE 2
Effect of proposed drug on DTH response

| | | Dosage, | 3P, % | |
|---|---|---|---|---|
| Group | Drug | mg/kg | (M ± m) | P |
| 1 control (intact) | | — | 20.0 ± 2.46 | — |
| 2 control (irradiated) | | — | 6.1 ± 1.21 | — |
| 3 irradiated, treated with the | Proposed | 0.4 | 7.9 ± 1.28 | <0.05 |
| drug | drug | 0.6 | 17.8 ± 2.94 | <0.01 |

P - relative to control groups

Anti-oxidant effect: Anti-oxidant effect of the proposed drug under peroxidation of lipids turned to be actually 10 times more intense than that of ascorbic acid. This fact is important for practical application of the proposed drug not only in dermal cosmetology, but in oncology as well.

Fertility effect: During studies of the effect of proposed drug on mutagenic properties (on a dominant lethal mutation model) the fertility and fecundity of test animals as compared to control ones was found. At the stage of mature spermatozoa the level of post-implantation mortality substantially reduced which is quite in line with literature data on anti-mutagen effect of placenta preparations.

Adaptogenic effect: This type of effect is one of the most desirable in the search for new medicines, as it enables influence on physiological processes under physical, thermal, chemical overloads, especially to prevent intoxication, burns, to increase tolerance of organism, etc. In the course of research of the proposed drug it was found that animals (mice) after pretreatment with a therapeutical dose (0.7 mg/kg) for 7 days can then survive from a toxic dose of poison (strychnine), lethal irradiation, pseudomonas infection. We studied the effect of proposed drug on mice life duration after administration of dose 1 mg/kg for 30 days. After such prophylaxis the mice lived 1.5 times longer at an average.

Anti-stress effect: In the study of anti-stress effect of the proposed drug we applied multidimensional evaluation methods of condition of various components of emotional stress resistance in rats (behavioral and visceral components). The results of this study demonstrate the ability of proposed drug to increase emotional stress resistance, thus, to prevent development of functional disorders of highest nervous activity, general immunity as well as of cardiac activity metabolic disorders. The most effective was dose 30 μg per 100 g.

Lactosenous effect: Practical application of the proposed drug in veterinary medicine showed that under agalaxia in sows the drug restores lactation process.

Anti-coagulant effect: In studies of the effect of proposed drug on blood we found that at dose of 0.15 mg/kg administered for one-month anti-coagulant effect was observed, but it disappeared within 30 days after suspension of administration. This fact may turn to be practically important in treatment of pre-infarction and post-infarction conditions.

Chondroprotective effect: Reference drugs in comparison for ability to restore cartilaginous tissue were such known chondroprotectors as rumalon, mucartrine and artheparone. Chondroprotective properties were evaluated on a standard model of posttraumatic coxa arthrosis in rats that developed after infliction of damage in the form of a round orifice 2 mm in diameter via articular cartilage to subchondral area. Such a damage brings to development of a posttraumatic arthrosis. In order to test the activity of the proposed drug and reference drugs dosages of 0.01; 0.03; 0.06; 0.1; 0.3; 0.5; 1.0 mg/kg of animal weight were selected. The effectivity of chondroprotection was evaluated along a specially developed scale, maximum mark being 12 scores which corresponds to complete resorption of connective tissue and restoration of cartilaginous tissue. The results of this evaluation in scores:
Rumalone—7.6
Mucartrine—8.4
Artheparone—8.9
Proposed drug—11.8.

Anti-inflammatory effect: This effect was tested on a model of experimental granuloma in rats. Reference drugs were those obtained on a basis of human placenta—PDS (placenta denaturated suspended) and amniocene. The drugs were administered subcutaneously at the time of operation. The test results are presented in Table 3.

TABLE 3
Results of study of proposed drug effect on proliferation and exudation

| | | | Reduction of | Reduction of |
|---|---|---|---|---|
| | | | proliferation, | exudation, % |
| | Number of | Dose, | % relative to | relative to |
| Drug | animals | mg/kg | control group | control group |
| Proposed drug | 10 | 1.0 | 38.7 ± 10.8 | 28.0 ± 10.9 |
| Amniocene | 11 | 10 | 26.2 ± 6.3 | 14.2 ± 3.6 |
| PDS | 8 | 50 | 33.5 ± 6.7 | 25.3 ± 4.8 |

Resorptive effect: Resorptive effect was studied on a model of commissure process appearing in non-pregnant female rats as a result of a dosed trauma of uterus horn. The results of tests are shown in Table 4.

TABLE 4
Results of study of proposed drug effect on commissure process in rats

| | Number | | | | |
|---|---|---|---|---|---|
| | of | Dose, | Commissure mass, % to control group | | |
| Drug | animals | mg/kg | 3 days | 10 days | 21 days |
| None | 15 | — | 57.4 ± 4.3 | 141.6 ± 37.8 | 36.5 ± 5.8 |
| (control) | | | | | |
| Proposed | 5 | 0.5 | 18.5 ± 3.6 | 16.8 ± 2.8 | 3.2 ± 0.9 |
| drug | | | | | |
| Proposed | 15 | 1.0 | 14.8 ± 3.7 | 12.5 ± 3.7 | 1.2 ± 0.8 |
| drug | | | | | |
| Amniocene | 15 | 20 | 19.2 ± 2.3 | 30.5 ± 6.2 | 7.0 ± 1.1 |
| ″ | 15 | 50 | 14.0 ± 1.9 | 21.6 ± 5.2 | 5.3 ± 0.9 |

Reparative effect: This type of pharmacological effect was studied on a standard model with a full-layer skin defect on mice who were administered the proposed drug in doses of 0.5-1.0 mg/kg. Under the effect of the drug healing process was reduced from 17 days in control group to 10 days. Amniocene, a placenta preparation, produced a similar effect only in dosage of 75 mg/kg. An important advantage of proposed drug is lack of scars in healing process.

The proposed drug was studied for acute and chronic toxicity in the full scope of WHO recommended methods.

Acute toxicity: Acute toxicity of the proposed drug was tested on mice by progressive (twice each time) increase of drug dosages, beginning from 1 mg/kg animal mass up to 100 mg/kg. At maximum acceptable dose (100 mg per 1 kg mass) the animals survived, which demonstrates absolute harmlessness of the drug. At high dosages, 5000 more than conditional therapeutic dose, the animals showed hypodynamia, apathy, flaccidity, but their activity was restored after 3-4 days without any signs of poisoning.

Chronic toxicity: Chronic toxicity of the proposed drug was tested in accordance with WHO recommendations on three groups of animals (rats, rabbits and dogs) within 6 months of annual administration of the drug in dosages that exceed conditional therapeutic value 10, 20, 50 and 100 times. After 6 months of administration 50% of animals were slaughtered in order to evaluate the effect of drug on organs and tissues, whereas other animals were rehabilitated for 1 month and then slaughtered for comparative evaluation of pathological effect of the drug. The study of internal organs of animals after 6 months of drug administration in the specified dosages showed that none of them died. The found changes in lungs, brain and kidneys were completely restored after one month of rehabilitation, which is indicative of complete harmlessness of the proposed drug (high bioavaliabiloity and biocompatibility).

Results of clinical tests: Clinical tests of the proposed drug were performed in three stages. First stage: 30 volunteers were intramuscularly administered aqueous solution of the proposed drug containing 1 mg of active substance within 10 days. No toxic or other side effects were found. At the second stage 90 people were administered in order to determine therapeutic dose of the drug as anti-inflammatory and analgesic for osteoarthrosis (one of promising areas of its biological effect). The third stage was performed in three Moscow clinics, total number of patients being 156 persons (male and female, age 18 to 78) with osteoarthrosis of various etiology and severity. The main clinical demonstration of osteoarthrosis is pain syndrome that was evaluated on a specially designed 5-score scale both before and after treatment. The proposed drug was administered parenterally and intra-articularly, the dose of active substance being 2 mg first every second day (10 injections), then every day (20 injections). Total dosage was 60 mg for each patient. The subjective evaluation of efficiency showed that pain substantially subsided in 58 patients (37%), totally disappeared in 42 (27%), whereas in 33 persons pain totally disappeared in knee joints and substantially subsided in coxofemoral joint. All patients enlarged the scope of their motions, particularly on stairs. 12 people delivered to hospital on crutches began walking on their own feet. No side effects were observed during the tests.

Post-clinical tests: Post-clinical tests of the proposed drug showed high efficiency under multiple pathological deviations, thus proving its bionormalizing effect. Some examples of such tests are given below.

### EXAMPLE 1

Patient L., age 33. Diagnosis: consequences of infantile cerebral paralysis (spasms of right hand and total arm, motional hypodynamia). After a course of treatment with the proposed drug for one year combined with physiotherapeutic methods, wherein the proposed drug was used as well, he made an actually complete recovery. The patient could write, peel potatoes and do other small motions with a high accuracy. He began running and swimming. No known drugs applied for 30 years before that gave any positive results.

### EXAMPLE 2

Application of proposed drug in infant and adolescent psychoneurological practice. Therapeutic correction of psychic and neurological syndromes that formed due to early age organic lesions of central nervous system, closed craniocerebral injuries and neuroinfections in infants and adolescents has remained a difficult problem up to appearance of the proposed drug. Application of the proposed drug for treatment of such conditions within 25-30 days in connection with physiotherapeutic methods at Ukrainian Research Institute of Infant and Adolescent Health ensured a high stable therapeutic effect (95.4%). The rest of patients also showed a substantial improvement of health condition.

### EXAMPLE 3

Application of the proposed drug in treatment of toxoplasmosis. Toxoplasmosis is a zootropic parasitary disease characterized in polymorph clinical pattern, latent, acute and chronic course after acquired or congenital infection. As a rule, toxoplasmosis cannot be effectively cured and becomes a prolonged chronic disease. The proposed drug tested by Bakterin Company attached to Dnepropetrovsk Medical Academy proved high pharmacotherapeutic effect, characterized by full recovery in most cases.

### EXAMPLE 4

Application of the proposed drug in treatment of chronic cerebral circulation disorders. After treatment of patients with diagnosed stroke, irrespective of remoteness, at Polyclinic #3, City of Kharkov, brain hematoma, commissures and other pathological defects completely resolved. Finally the lost motion function and speech abilities were restored. The duration of treatment depended on severity and longevity of pathological condition.

### EXAMPLE 5

Efficiency of urogenital infection treatment. High therapeutic effect was obtained from application of the proposed drug in treatment of such diseases as chlamidiosis, ureoplasmosis, etc., especially of their combinations. Tests at urological clinic of Kharkov Medical University gave positive effect in 94.7% of cases.

### EXAMPLE 6

Application of the proposed drug in cardiology. In Polyclinic #6, City of Kharkov, above 200 patients with cardiovascular insufficiency (post-infarction condition, angina pectoris, myocarditis, etc.) were treated. The absolute majority of patients had a sustained therapeutic effect, especially those with post-infarction condition and angina pectoris.

### EXAMPLE 7

Application of the proposed drug in treatment of traumas and diseases of locomotive system. Treatment of patients with various etiology diseases of locomotive system at Polyclinic #6, City of Kharkov, gave positive effect in actually 100% of cases.

### EXAMPLE 8

Application of the proposed drug in gynecological practice. High efficiency of treatment of such gynecological diseases as endometritis, diffuse fibromyoma, adnexitis, cystic disease, adnexa inflammation, etc. was shown in tests at some gynecological clinics of Kharkov.

The drug has also proved its effectiveness in oncology, ophthalmology, surgery and other areas of medicine. The drug under trademark Bioglobine-U was permitted for medical application (Registration Certificate UA #3273/01/01 of 24.06.2005, Ukraine).

### EXAMPLE 9

Application of the proposed drug in agriculture. Biocatalytic effect of modulator on seeds and extra-radical parts of plants accelerates biochemical reactions in the process of photosynthesis during development, blossoming and fruit formation. This factor leads to growth of length and thickness of elevated part of plants, increased number of stems, inflorescences and foliage area and, respectively, to increased yield of all treated cultures. This effect embraces actually all types of plants, including houseplants, weeds and fruit trees. Such broad action is caused by unity between live nature (plants) and the modulator and ensures its existence.

Adaptogenic and immunotropic activity of the proposed modulator ensures resistance of plants to ground frost and drought. The results of observation at certain domestic gardens showed that tomato stems that fell and blackened due to frost after treatment with modulator renewed their growth, development and adequate fruit formation. A similar effect was observed during drought. The yield increased as compared to control sections by 25 to 200% depending on environment conditions.

Due to its adaptogenic and immunotropic activity proposed modulator increases resistance of fruits to infections both during their growth and during storage.

One of substantial advantages of the proposed plant growth and development modulator as compared with known substances is environmental purity of obtained products and elimination of negative effect on environment, as the plants and modulator have identical biochemical nature.

In practice the proposed modulator is administered by presowing treatment of vegetable seeds or tubers with 0.0005-0.005% solution of the drug (as per active substances) made as 2 ml of commercial Bioglobine (1 teaspoonful) per 10 L of water. At small household gardens they take 5-10 drops of commercial product per 1 cup of water. Seeds are treated before planting for 24 hours, whereas tubers for 10-15 minutes.

Extra-radical treatment of plants or fruit trees is effected by spraying with solution of commercial Bioglobine in the above concentrations at the period of sprouting, budding and blossoming.

The results of testing of proposed plant growth and development modulator under field conditions performed at Institute of Vegetable, Watermelon and Melon Growing, Ukrainian Academy of Agrarian Science, at Kharkov Vegetable Factory and at household gardens of some natural persons, are presented in Tables 5-9.

TABLE 5
Yield of modulator-treated potatoes
(Institute of Vegetable, Watermelon and Melon Growing,
Ukrainian Academy of Agrarian Science)

| | | | | | Yield of |
|---|---|---|---|---|---|
| | Time of | | | Yield | seed |
| | treatment | | Yield, | relative to | fraction |
| Option | (phenological | Concentration, | metric | control | tubers, |
| # | phase) | mg/L | centners | section, % | % |
| 1 | Before planting | 0.5 | 147.4 | 96.8 | 76 |
| 2 | | 2.5 | 171.9 | 112.9 | 70 |
| 3 | | 5.0 | 175.3 | 115.2 | 79 |
| 4 | Sprouting | 0.5 | 185.2 | 121.7 | 80 |
| 5 | | 2.5 | 153.8 | 101.0 | 78 |
| 6 | | 5.0 | 173.7 | 114.1 | 76 |
| 7 | Budding | 0.5 | 184.0 | 120.9 | 80 |
| 8 | | 2.5 | 207.1 | 132.5 | 73 |
| 9 | | 5.0 | 194.0 | 127.5 | 82 |
| 10 | Blossoming | 0.5 | 177.6 | 116.7 | 78 |
| 11 | | 2.5 | 127.5 | 83.8 | 71 |
| 12 | | 5.0 | 124.3 | 81.7 | 75 |
| 13 | Combination | 0.5 | 143.6 | 94.3 | 79 |
| 14 | | 2.5 | 153.1 | 100.6 | 74 |
| 15 | | 5.0 | 140.7 | 92.4 | 68 |

TABLE 6
Biometric parameters of modulator-treated potatoes
(Institute of Vegetable, Watermelon and Melon Growing,
Ukrainian Academy of Agrarian Science)

| | | | | | Average | |
|---|---|---|---|---|---|---|
| | Time of | | No of seeds | | mass of | Average |
| | treatment | | | 000 | seed- | number |
| | (phenological | Concentration, | | per | bearing | of stems |
| Option # | phase) | mg/L | per bush | hectare | potatoes | per bush |
| 1 | Before planting | 0.5 | 6.6 | 269 | 58.6 | 6.9 |
| 2 | | 2.5 | 6.4 | 261 | 50.3 | 8.1 |
| 3 | | 5.0 | 7.5 | 306 | 47.8 | 6.7 |
| 4 | Sprouting | 0.5 | 6.4 | 261 | 58.9 | 8.2 |
| 5 | | 2.5 | 6.2 | 253 | 50.2 | 7.9 |
| 6 | | 5.0 | 7.1 | 290 | 49.6 | 7.0 |
| 7 | Budding | 0.5 | 7.7 | 314 | 49.3 | 8.0 |
| 8 | | 2.5 | 7.2 | 294 | 54.7 | 7.4 |
| 9 | | 5.0 | 7.4 | 302 | 55.7 | 8.0 |
| 10 | Blossoming | 0.5 | 6.7 | 273 | 54.7 | 6.7 |
| 11 | | 2.5 | 4.6 | 188 | 52.0 | 6.6 |
| 12 | | 5.0 | 4.6 | 188 | 51.3 | 9.2 |
| 13 | Combination | 0.5 | 5.3 | 216 | 55.5 | 8.2 |
| 14 | | 2.5 | 6.7 | 273 | 45.1 | 7.9 |
| 15 | | 5.0 | 5.5 | 224 | 47.3 | 8.1 |

| | | | | | |
|---|---|---|---|---|---|
| 16 | Control (no treatment) | 5.7 | 233 | 53.2 | 8.2 |

TABLE 7
Virus affection of modulator-treated potatoes

| | Time of | | | | | | | | |
|---|---|---|---|---|---|---|---|---|---|
| | treatment | | Virus affection of plants, % | | | | | | |
| | (phenological | Concentration, | | Latent form, including | | | | | |
| Opt. # | phase) | mg/L | Open form | X | S | M | Y | F | Total |
| 1 | Before | 0.5 | 0.9 | 0 | 10 | 30 | 0 | 12 | 30 |
| 2 | planting | 2.5 | 0.8 | 0 | 0 | 10 | 0 | 0 | 14 |
| 3 | | 5.0 | 0.8 | 2 | 12 | 6 | 2 | 7 | 20 |
| 4 | Sprouting | 0.5 | 1.2 | 2 | 0 | 30 | 10 | 9 | 36 |
| 5 | | 2.5 | 2.4 | 0 | 0 | 10 | 0 | 14 | 12 |
| 6 | | 5.0 | 1.3 | 2 | 2 | 16 | 0 | 2 | 20 |
| 7 | Budding | 0.5 | 1.8 | 2 | 2 | 12 | 2 | 6 | 12 |
| 8 | | 2.5 | 1.4 | 4 | 4 | 10 | 0 | 12 | 18 |
| 9 | | 5.0 | 0.4 | 0 | 0 | 20 | 0 | 2 | 20 |
| 10 | Blossoming | 0.5 | 0.5 | 2 | 0 | 10 | 0 | 0 | 12 |
| 11 | | 2.5 | 0 | 6 | 2 | 14 | 10 | 2 | 20 |
| 12 | | 5.0 | 0.4 | 6 | 2 | 24 | 10 | 6 | 32 |
| 13 | Combination | 0.5 | 1.3 | 0 | 2 | 20 | 0 | 2 | 24 |
| 14 | | 2.5 | 2.6 | 4 | 2 | 24 | 2 | 6 | 28 |
| 15 | | 5.0 | 3.4 | 2 | 12 | 0 | 22 | 4 | 28 |

| | | | | | | | | |
|---|---|---|---|---|---|---|---|---|
| 16 | Control (no treatment) | 1.1 | 1 | 3 | 9 | 17 | 10 | 29 |

TABLE 8
Productivity and its components of sweet pepper
under various treatment options

| | | | Average | |
|---|---|---|---|---|
| | Treatment | Plant | number of | Average mass |
| Breed | option | productivity, g | fruits | per fruit, g |
| Nadezhda | 1 | 678.00 | 9.17 | 73.95 |
| | 2 | 1355.00 | 16.00 | 85.05 |
| | 3 | 422.00 | 8.90 | 44.81 |
| Gift of | 1 | 852.00 | 9.79 | 87.00 |
| Moldova | 2 | 650.50 | 10.51 | 61.72 |
| | 3 | 590.50 | 8.55 | 69.11 |
| HIP₀₅ | | 38.92 | 0.78 | 5.94 |

Under field crop rotation we studied the effect of proposed drug on development of beetroots (breed Diy), radishes (breed Xenia), parsley (breed Kharkovchanka), carrots (breed Yarkaya). The seeds were wetted for 24 hours in a solution of 0.5 mg/l of the drug.

Such options were tested:
I—dry seeds
II—wetting in water
III—wetting in Bioglobine solution.

TABLE 9
Effect of modulator on quantity and mass of root crops
of beetroots, radishes, parsley and carrots

| | | | | Non- | | | |
|---|---|---|---|---|---|---|---|
| | | Standard | | standard | | Undersize | |
| | | | Mass, | | Mass, | | Mass, |
| Vegetable | Option | Number | kg | Number | kg | Number | kg |
| Beetroots | I (control) | 11 | 3.8 | 9 | 0.58 | 7 | 0.32 |
| | II | 14 | 6.7 | 11 | 0.82 | 9 | 0.30 |
| | III | 23 | 10 | 4 | 0.68 | 3 | 0.10 |
| Radishes | I (control) | 42 | 0.40 | 34 | 0.32 | 47 | 0.20 |
| | II | 29 | 0.25 | 37 | 0.29 | 52 | 0.18 |
| | III | 37 | 0.45 | 59 | 0.41 | 63 | 0.2 |
| Parsley | I (control) | 29 | 1.4 | 12 | 0.45 | 23 | 0.32 |
| | II | 30 | 1.75 | 13 | 1.1 | 29 | 0.78 |
| | III | 57 | 4.3 | 10 | 0.8 | 17 | 0.25 |
| Carrots | I (control) | 36 | 3.4 | 27 | 1.3 | 74 | 1.2 |
| | II | 35 | 3.9 | 41 | 1.9 | 65 | 1.05 |
| | III | 54 | 7.8 | 28 | 1.15 | 44 | 1.2 |

Regulating effect of the proposed drug should be noted as regards formation of first year cabbage biometric parameters. The drug-treated plants showed much longer stem as well as surface area of the second and third true leaves. The best result in reduction of undersize leaves was obtained under maximum drug concentration (5 mg/L): 80.3% for breed Kharkovskaya zimnyaya and 78.6% for breed Yaroslavna. Control values were 43.9% and 37.9% respectively. It must be noted that acclimating rate of meristematic material of white cabbage, as of any vegetable, when exacted from aseptic planting conditions has always been low.

In this aspect we must specify a positive role of the proposed drug in stimulation of adaptogenic action of test-tube plants to in vivo planting conditions.

When planting breed Kharkovskaya zimnyaya cabbages in a glass greenhouse the increase of leaf surface was 1.6 times higher as compared to control section, 95.8% of incepted heads reached commercial maturity, whereas in control section this value was 56.7%. For breed Yaroslavna the increase of leaf surface was 1.33 times higher and 98% of cabbage heads had commercial maturity (45.1% at control section). It should be noted that cabbages were grown on closed soil during all summer under high day temperatures (up to 35-40° C.), which is not typical in growing of this vegetable in open soil. In this aspect the positive role of the proposed drug should be specified as a regulator increasing plant resistance to that abiotic stress.

The results of the proposed drug efficiency showed in the tables above were obtained in poorly tended fields. If fields are correctly structured, yield of potatoes, cabbage and other cultures treated with modulator will increase more than 2 times. Tests at Kharkov Vegetable Factory proved that yield of cucumbers and tomatoes after three treatments (during sprouting, budding and blossoming) increased by 2-2.5 kg per square meter.

The proposed drug turned to be highly efficient in treatment of grapes during budding, blossoming and initial phase of pea formation. Yields of grapes provided duly fertilized increased more than 4 times. Partially such effect is caused by special physico-chemical and biochemical properties (fertile effect) of the product as noted above. Raw materials in this case were floral biopolymers instead of placenta, such as soy flour.

The closest known solution to this invention is “Bionormalizing-Effect Drug and Method of Manufacturing Same” (RU 2235550 of 10.09.2004). Therein the final product as isolated after chemical treatment of placenta tissues with chlorous acid is a complicated mix of biochemical substances, the basic active substance not being identified.

The basic quality parameters of the prototype product are bioactivity and polypeptide content. Therapeutic dose of the drug as established by testing is 2 ml of solution with average content of 40 mg dissolved matter, including at least 1 mg polypeptides. Biological activity is evaluated by drug ability to increase the activity of blood neutrophil lysosome membrane enzymes and to enhance energy and oxidation metabolism. Manufacturer was determined by a standard WHO methodology, i.e. by response reaction of neutrophils in heparinized mice blood that was demonstrated in spontaneous reduction of nitro-blue tetrazolium (HCT test).

Biological activity of the prototype drug was at least 50,000 units per 1 ml of drug. In spite of high pharmaco-therapeutic results of application of prototype drug, the method of its manufacture has a number of substantial disadvantages due to inadequate engineering, namely:
presence of multiple components, mostly unnecessary ones, which, though not toxic, may produce negative effect by inhibiting the activity of basic active substance;
novel components of the product, such as polypeptides, amino acids, hexuronic acids and amino sacharides, are not characterized quantitatively and qualitatively, particularly, as regards their degree of oxidation;
the structure of basic active substance is not established, its basic features causing the positive effects are not determined;
the method of drug manufacture does not ensure high yield of basic active substance, thus reducing drug biological activity, especially its capacity as a modulator of metabolic processes.

Those disadvantages are eliminated by our additional research and development of a new method for manufacture characterized by application for chemical modification of biopolymers of an oxidizing, chlorinating and hydrolyzing mixture extracted after ion exchange treatment of chlorous acid salts with sulfuric cationite in H form:

R SO₃H+NaClO₂→R SO_{c}Na+HClO₂

Under the effect of sulfuric cationite chlorous acid suffers chemical transformation:

9 HClO₂→3 HCl+3 H₂O+3 ClO₂+1.5 Cl₂

The main obstacle for extraction of active substance from reaction mixture in the prototype process was incomplete desorption of final product during ion exchange. This obstacle was removed by preliminary fine milling of sulfopolystyrene cationite type KY-2-8, Lewatit, Dowex, etc. in H form at Dezi II ML 1F disintegrator (Estonian-made) having two counter-rotating gearwheels with velocity 12,000 rpm. Particle size after milling was less than 1-2 μm. Below an example of particular embodiment of the proposed method is given.

### EXAMPLE 10

Minced and purified from blood human or animal placenta is loaded to a titanium reactor having an agitator and coil for heating or cooling. A prepared mixture was added containing sodium salt of an amino acid (preferably lysine) and cobalt chloride in equivalent relation 0.001-0.1% of lysine cobalt salt to dry residue of minced placenta. Then a respective quantity of oxidizing-chlorinating mixture obtained by treatment of 5% solution of chlorous acid sodium salt with sulfocationite in H form is transferred to the reactor so than 0.1-1.5 parts of chlorous acid sodium salt taken for sorption related to 1 part of dry placenta and allowed to stand for 1-45 days at temperature of 18-60° C. up to necessary degree of chemical modification.

The degree of chemical treatment completeness is determined by percentage of chlorine in water-soluble reaction products; it mist be 0.5 to 40%. When the necessary degree of chemical treatment is attained, the process is suspended, the heterogeneous system of reaction mixture is separated, liquid phase is subjected to ion exchange purification in order to obtain medical forms of the drug (ampoulized solution or lyophilized powder).

To obtain the final product for agricultural application the reaction mixture after attainment of necessary chemical modification degree is neutralized with an alkaline agent and filled to containers in the form of slurry.

Then the liquid phase is treated with active carbon and passed through an ion exchange column filled with pre-milled resin. To reduce resin resistance in the process of sorption and desorption milled resin in H form is mixed with non-milled cationite in Na form in relation 1:1 or 1:2 depending on the fineness of milling.

The final product is eluded with 1 N caustic soda solution and extract fractions at pH value from 11.5 to the moment when alkali “overshoot” begins. To the purpose of finer separation of final product fractions are extracted stepwise at each increase of 0.5 of pH value. The extracted fractions are qualitatively analyzed by IR spectrophotometer according to known method (Kullbom S. D., Smith H. F., Anal.Chem., 1963, 35, p. 1005). In accordance with the results of analysis fractions of final products are united depending of sulfogroup and chlorine content (Schoeniger method).

The fraction extracted at pH 11.5-14.0 is neutralized by hydrochloric acid solution up to pH=7.0, biological activity of final product solution is determined by HCT test, and the solution is diluted with distilled water up to a therapeutic dose corresponding to 50,000 IU per 1 ml of neutralized solution. The diluted solution is subjected to sterilizing filtration and transformed to medical injection form of ampoulized solution or lyophilized powder by known technique.

The examples are to be construed as merely illustrative and not limitative of the remainder of the disclosure in any way.

While the foregoing written description of the invention enables one of ordinary skill to make and use what is considered presently to be the best mode thereof, those of ordinary skill will understand and appreciate the existence of variations, combinations, and equivalents of the specific exemplary embodiments thereof. The invention is therefore to be limited not by the exemplary embodiments herein, but by all embodiments within the scope and spirit of the claims.

1. Modulator of metabolic processes on the basis of chemically modified biopolymers with a polypeptide chain, characterized in that the final product comprises chlorine atoms covalent-bonded with nitrogen atoms of polypeptide chain to the amount of 0.5% to 40% of the total mass of modified biopolymer.

2. Modulator of metabolic processes as per claim 1 on the basis of chemically modified biopolymers with a polypeptide chain, characterized in that the polypeptide chain includes methionine sulfoxide, cysteinic, α-aminomalonic, α-aminoacetoacetic and cysteinic acids to the amount of 1-20 acid residues per 1 mole of final product.

3. Method of preparation of modulator as per claims 1-2 by chemical modification of natural biopolymers with polypeptide chain, characterized in that chemical modification comprises processes of chlorination, oxidation and hydrolysis.

4. Method as per claim 3, characterized in that in chemical modification an oxidizing, chlorinating and hydrolyzing mixture is used as obtained after treatment of chlorous acid salts with a sulfocationite under static conditions.

5. Method as per claim 3, characterized in that in the process of biopolymer chemical modification with oxidizing, chlorinating and hydrolyzing mixture the mass relation between biopolymer and chlorous acid salt taken for ion exchange is 1: (0.1-1.5).

6. Method as per claim 3, characterized in that the process of chemical modification is performed at temperature 18-60° C.

7. Method as per claim 3, characterized in that final product is extracted from reaction mixture by sorption with a sulfuric acid cationite in H form, previously finely milled, with subsequent fractional desorption with caustic soda solution, the process being controlled by chlorine and sulfogroup content in final product, and finally the product is transferred to a medicinal drug form by known techniques.

8. Method as per claim 3, characterized in that the final product for agricultural application is transferred to operable form as slurry by neutralization of reaction mixture after chemical modification with an alkaline agent.
